(19) United States
(12) Patent Application Publication
(10) Pub. No.: US 2006/0122541 A1
(43) Pub. Date: Jun. 8, 2006

(54) Adjusting the length of the leg

(76) Inventor: Gregor Tuma, Munchen (DE)

Correspondence Address: RENNER, OTTO, BOISSELLE & SKLAR, LLP, Nineteenth Floor, 1621 Euclid Avenue, Cleveland, OH, 44115-2191, US

(21) Appl. No.: 11/269,143

(22) Filed: Nov. 8, 2005

Related U.S. Application Data
(60) Provisional application No. 60/645,149, filed on Jan. 19, 2005.

(30) Foreign Application Priority Data
Nov. 8, 2004 (EP) 04026471.5

(51) Int. Cl. A61B 5/107; A61F 2/36

(52) U.S. Cl. 600/587; 606/102; 128/922; 623/23.14; 623/23.26; 623/23.42

(57) ABSTRACT

A method and device for selecting a hip joint prosthetic element from a number of hip joint prosthetic elements, wherein bone and/or cartilage contour data of the pelvis and/or the femur are detected and at least one hip joint prosthetic element is selected based on the detected bone and/or cartilage contour data. The device for selecting at least one hip joint prosthetic element from a number of hip joint prosthetic elements, includes a data detection system for detecting bone and/or cartilage contour data, a computational unit for processing the detected bone and/or cartilage contour data, wherein the computational unit is connected to the data detection system, and a database in which characteristic data of a number of hip joint prosthetic elements are stored. The computational unit selects at least one hip joint prosthetic element from the database in accordance with the detected bone and/or cartilage contour data.

### RELATED APPLICATION DATA

This application claims priority of U.S. Provisional Application No. 60/645,149 filed on Jan. 19, 2005, which is incorporated herein by reference in its entirety.

### FIELD OF THE INVENTION

The present invention relates to a device and a method for selecting at least one hip joint prosthetic element from a number of hip joint prosthetic elements in hip joint operations. More particularly, the invention relates to selecting the at least one hip joint prosthetic element based on a contour of the pelvis and/or the femur, wherein differences in the leg length and leg offset are taken into account and/or compensated.

### BACKGROUND OF THE INVENTION

In a known method for selecting at least one hip joint prosthetic element and/or a hip joint prosthetic, a surgeon, prior to the operation, produces a representation of the hip to be operated on based on x-ray recordings. From this representation, the model and size of the hip joint prosthetic are selected.

### SUMMARY OF THE INVENTION

In a method for selecting at least one hip joint prosthetic element from a number of hip joint prosthetic elements, bone and/or cartilage contour data of the pelvis and/or the femur may be detected, wherein at least one hip joint prosthetic element can be selected based on the detected bone and/or cartilage contour data. In particular, the shape and/or contour of the pelvis and/or the femur can be detected, for example, using a computer-assisted navigation system, wherein the amount of removed bone and/or cartilage material can be measured. Before the bone and/or cartilage contour data are detected, markers, such as reference stars, for example, can be attached to the hip and/or the femur to register a bone and/or cartilage contour or a shape of the pelvis and/or the femur.

In the registering process, coordinate systems can be defined, for example, by sensing or scanning landmarks (e.g., characteristic positions or points of the hip and/or the femur), wherein the structure, shape or contour of the hip and/or the femur can be registered or defined. The coordinate system of the hip to which the contour of the bone and/or the cartilage of the pelvis may be registered or defined, can be defined by the front hip plane and the mid-sagittal plane, for example. The coordinate system of the femur can be defined such that the direction of the leg length and/or the direction of the leg offset can be registered or defined with respect to the femoral coordinate system. This enables a neutral position of the femur and in particular a neutral position of the femur relative to the hip to be virtually ascertained, for example, from the direction of the leg length and/or the direction of the leg offset.

At least one hip joint prosthetic element can be ascertained or selected before and/or during a hip joint operation, for example, from the detected shape or contour of the bones, cartilage of the pelvis, femur, and/or from the detected missing and/or removed bone and/or cartilage material, wherein the hip joint prosthetic element can be a prosthetic head, a prosthetic shaft and/or a prosthetic cavity. By taking into account the removed bone material, suitable hip joint prosthetics, which enable compensation for differences in the leg length and/or the leg offset, can be simply, quickly, automatically and exactly ascertained.

The bone and/or cartilage contour data can be detected using a pointer (e.g., the hip and/or the femur can be scanned with the pointer). Alternatively, the hip and/or the femur can be optically scanned before and/or during a hip joint operation, for example. Each technique can be used to obtain the contour or shape of the hip and/or the femur, which then can be registered with respect to particular coordinate systems.

The bone and/or cartilage contour data also can be detected and/or registered using a recording method such as a computer tomography method, a nuclear spin tomography method, an ultrasound method, a positron emission tomography method (PET) and/or a single photon emission computed tomography method (SPECT).

In particular, a first set of bone and/or cartilage contour data can be detected and/or registered before and/or during a hip joint operation to obtain a pre-operative or intra-operative shape or contour of the pelvis and/or the femur, for example. From the shape and/or contour, conclusions can be drawn regarding differences in the leg length and/or the leg offset, from which an amount of removed bone and/or cartilage material can be ascertained, for example. The detected bone and/or cartilage contour data can be provided to a navigation system (e.g., via direct input or via transmission by a network or other wired or wireless transmission method), wherein the navigation system can calculate a neutral standing position of a person from the detected and/or registered bone and/or cartilage contour data. In this way, the direction of the leg length of the femur can be aligned parallel to the front hip plane and/or to the mid-sagittal plane. This can provide a person in a standing position with legs running at least approximately parallel to each other, and/or the hip of the person lay at least approximately parallel to a standing surface of the person.

Data, such as an amount of bone and/or cartilage material that should be or has been removed, also could be input into the navigation system. Using this data, the navigation system, based on detected or registered bone and/or cartilage contour data, can navigate an instrument for removing the bone and/or cartilage material. While the bone and/or cartilage material of the hip and/or the femur is being removed and/or after it has been removed by means of an instrument navigated by the navigation system, for example, a second set of bone and/or cartilage contour data can be detected and/or registered.

For example, the second set of bone and/or cartilage contour data can be detected and/or registered using a pointer and/or an imaging method, wherein at least one hip joint prosthetic element, such as a prosthetic head, a prosthetic cavity or a prosthetic shaft, is selected based on the first set of bone and/or cartilage contour data obtained before and/or during the hip joint operation, and on the second set of bone and/or cartilage contour data ascertained during and/or after the hip joint operation.

The hip joint prosthetic element can be selected from the difference between the first and second set of bone and/or cartilage contour data, e.g., from the difference in the contour of the pelvis and/or the femur before and after the hip joint operation, such that the selected hip joint prosthetic element compensates for the difference between the pelvis and/or the femur before and after the operation, wherein the difference may be created by the removed bone and/or cartilage material. Preferably, the hip joint prosthetic element can be selected such that it compensates for or obtains a leg offset prior to the operation or differences in the leg length prior to the operation, or such that the pre-operative data are taken into account when selecting the hip joint prosthetic element, in addition to the bone and/or cartilage material removed by the navigated instrument.

The amount of the bone and/or cartilage material removed by the navigated instrument also can be ascertained during the removal process, for example, by detecting the positional data of the instrument and processing the data using a computational unit to determine the amount of bone and/or cartilage material that has been removed. Preferably, the hip joint prosthetic element can be selected such that the leg length and/or the leg offset, which may be predetermined or known before the hip joint operation, is obtained based on a difference in the leg length prior to the operation, an existing leg offset and/or removed or missing bone and/or cartilage material when selecting the at least one hip joint prosthetic element.

Furthermore, the invention provides a computer program which, when it is loaded onto a computer or is running on a computer, performs a method as described above. The invention further provides a program storage medium or a computer program product comprising such a program.

In order to select at least one hip joint prosthetic element from a number of hip joint prosthetic elements, a device for selecting the at least one hip joint prosthetic element preferably includes: a data detection apparatus for detecting bone and/or cartilage contour data, such as the shape, contour or outer structure of the pelvis and/or the femur; a computational unit connected to the data detection system, wherein the computational unit processes the detected bone and/or cartilage contour data transmitted from the data detection system to the computational unit (e.g., via a wire connection such as via a LAN network and/or wirelessly, for example by means of Bluetooth, WLAN or HIPERLAN); and a database or database memory or memory system such as a bulk memory, in which characteristic data of a number of hip joint prosthetic elements can be stored. The computational unit can select at least one hip joint prosthetic element from the database or memory in accordance with detected bone and/or cartilage contour data, for example, by comparing the detected bone and/or cartilage contour data, such as the contour of the pelvis and/or the femur, with a number of data for hip joint prosthetic elements available in the database or memory, such as the contour or shape of various hip joint prosthetic elements. The computational unit can select at least one hip joint prosthetic element from the database or memory that has the greatest match or similarly to the detected bone and/or cartilage contour data, for example.

The data detection system preferably can comprise at least one pointer provided with markers, and at least one camera, such as an infrared camera, and/or infrared lamps, wherein the markers can be active markers emitting infrared radiation or passive markers reflecting infrared radiation. The emitted or reflected infrared radiation can be detected by the at least one camera, such that the position of the pointer and therefore the contour of the pelvis and/or the femur can be deduced. The data detection system also can comprise an instrument provided with markers such that the instrument can be detected by the cameras, wherein the instrument can remove bone and/or cartilage material, wherein the amount of the removed bone and/or cartilage material can be deduced based on the position of the instrument. The contour or the shape of the pelvis and/or the femur also can be detected or registered using a computer tomograph, a nuclear spin tomograph, an ultrasound tomograph, a positron emission tomograph and/or a SPECT tomograph, each of which can form part of the data detection system.

The device for selecting at least one hip joint prosthetic element from a number of hip joint prosthetic elements preferably comprises a data output device, such as a screen or the like, which can graphically output the bone and/or cartilage contour data (e.g., as numerical values), and/or the selected hip joint prosthetic element (e.g., a number identifying the respective hip joint prosthetic element or the hip joint prosthetic element itself). A data input device, such as, for example, a keyboard and/or a scanner (e.g., a 3D scanner) also can be provided and can be connected to the computational unit, the database and/or memory. Using the data input device, data such as data for various known hip joint prosthetic elements can be input, or known hip joint prosthetic elements themselves can be scanned in and stored in the database or memory, for example. Further, data such as detected bone and/or cartilage contour data can be input that can be processed in the computational unit and then can be compared with known data from the database.

The device can comprise a navigation system that can be connected to the computational unit and, for example, can navigate a trackable instrument (e.g., an instrument that includes trackable markers) for removing bone and/or cartilage material. The instrument can be navigated based on the detected bone and/or cartilage contour data and the detected shape or contour of the pelvis and/or the femur, for example, by ascertaining how the instrument should be navigated, to what positions the instrument should be guided, or what movements the instrument should perform, in order to remove the desired amount of bone and/or cartilage material.

In a preferred embodiment, the selected hip joint prosthetic element, via the device, can be automatically positioned at a point, such as on the pelvis or on or in the femur, for example.

The selected hip joint prosthetic element, which can be both a prosthetic cavity and a prosthetic head or a prosthetic head connected to a prosthetic shaft, comprises a partially spherical surface element. The surface element can be at least approximately hemispherical, can be a spherical segment, a spherical sector and/or can be described as a spherical segment or spherical sector of a hollow sphere. An outer contour and an inner contour of the surface element can be partially spherical or at least approximately hemispherical. By inserting an additional element, for example, into the inner contour of the prosthetic head or onto the outer contour of the prosthetic cavity, the diameter and/or radius of the inner contour or outer contour of the hip joint prosthetic element can be varied. If the additional element is inserted into the inner contour of the prosthetic head, then the diameter of the convex inner contour of the prosthetic head is reduced by twice the thickness of the additional element and/or the radius of the inner contour of the prosthetic head is reduced by the thickness of the additional element. The additional element also can be placed on the outer contour of the prosthetic cavity, such that the diameter of the concave outer contour of the prosthetic cavity is increased by an amount corresponding to twice the thickness of the additional element.

The additional element also can be a removable element which may be removed from the hip joint prosthetic element or the inner or outer contour of the hip joint prosthetic element, wherein the diameter of the outer and/or inner contour of the hip joint prosthetic element can be varied by removing the additional element. The additional element, for example, can be removed from the inner contour of the prosthetic head, such that the diameter of the inner contour of the prosthetic head is increased by twice the thickness of the additional element which has been removed. The additional element also can be removed from the outer contour or the surface of the prosthetic cavity, such that the diameter of the outer contour of the prosthetic cavity is reduced by twice the thickness of the additional element. The additional element can exhibit various thicknesses, e.g., it can be one, three or five mm thick, or it can be up to 20 mm thick.

By inserting or removing the additional element, compensations can be made for differences in the leg length or the leg offset. Using the method described herein, a hip joint prosthetic element can be selected that may be provided with an additional element to compensate for or establish an existing, ascertained or desired difference in the leg length or leg offset.

The additional element also can be inserted into or attached to the prosthetic shaft, which can be connected to the prosthetic head, for example, or the additional element can be removed from the prosthetic shaft, wherein the length of the prosthetic shaft can be altered by removing or adding the additional element. The additional element can be placed on the prosthetic shaft or inserted into the prosthetic shaft, for example, such that adding the additional element increases the length of the prosthetic shaft by the length of the additional element, or the additional element can be removed from the prosthetic shaft, such that the length of the prosthetic shaft can be reduced by the length of the additional element. The additional element can be any length up to 20 mm, e.g., one, three, five or more mm in length.

The forgoing and other embodiments of the invention are hereinafter discussed with reference to the drawings.

### BRIEF DESCRIPTION OF THE DRAWINGS

FIG. 1 illustrates an exemplary device in accordance with the invention, as well as a hip and a femur.

FIG. 2 illustrates a hip and a femur, before and after removing bone and/or cartilage material.

FIG. 3 illustrates a femur before and after removing bone and/or cartilage material, and a hip before and after removing bone and/or cartilage material, as well as instruments for removing bone and/or cartilage material.

FIG. 4A illustrates a femur with an inserted prosthetic.

FIG. 4B illustrates a hip joint prosthetic.

FIG. 5 illustrates an exemplary flow diagram of a method in accordance with the invention.

FIG. 6 is a block diagram of a computer system that can be used to implement the method of the present invention.

### DETAILED DESCRIPTION

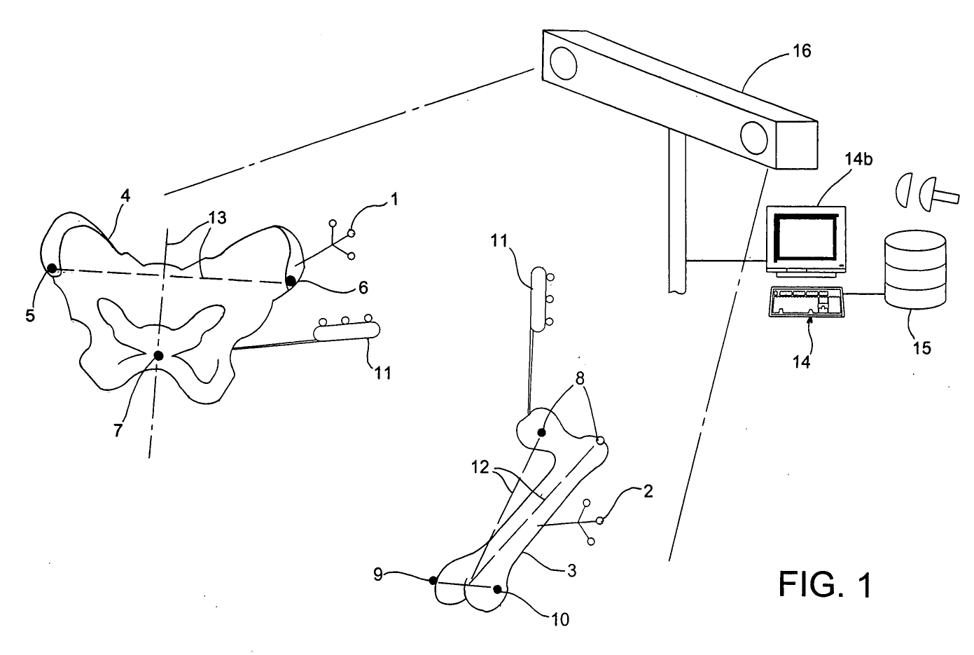

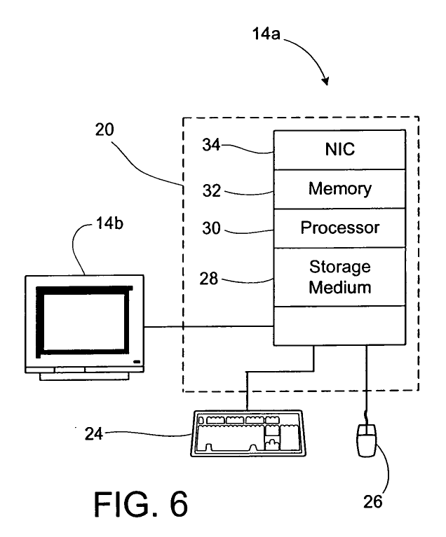

FIG. 1 shows an exemplary device for selecting a hip joint prosthetic element from a number of hip joint prosthetic elements, wherein the device includes a navigation system 14 (which can include an integral or separate computational unit 14a shown in FIG. 6), a screen 14b, a database 15 and a camera 16 (e.g., an infrared camera). A reference star 1, 2 is attached to each of a hip 4 and a femur 3, whereby the hip 4 and the femur 3 can be detected and registered by means of the camera 16, such that the position of the hip 4 and the femur 3, e.g., their precise spatial position, is known to the navigation system 14. In particular, the hip 4 and the femur 3 can be registered with the aid of known points such as landmarks 5, 6, 7, 8, 9, 10, for example, by identifying the landmarks to the navigation system 14, wherein the navigation system 14 then detects and registers the surface of the hip 4 (e.g., the hip joint cavity) and the femur 3 (e.g., the femoral head). The landmark points 5, 6, 7, 8, 9, 10 can be identified using a trackable pointer 11 (e.g., a pointer that include trackable markers attached thereto), wherein, for example, a tip of the trackable pointer is placed on the landmark points. Alternatively, the landmarks can be identified optically, e.g., using a laser to scan a surface of the hip to identify and/or detect the landmark points. In the computational unit 14, a neutral standing position of a person and/or a predetermined difference in the leg length and/or a predetermined leg offset can be calculated from the detected data, such as the bone and/or cartilage contour data or the shape of the hip 4 and/or of the femur 3. The hip joint cavity and/or the head of the femur is registered using the pointer 11, for example, such that the precise shape or contour of the hip joint cavity and/or the head of the femur 3 can be digitally displayed via the computational unit and/or via the navigation system 14. Thus, a neutral position of the hip 4 and the femur 3 can be simulated with respect to each other. Also, a neutral standing position of a person or a predetermined difference in the leg length and/or a predetermined leg offset also can be simulated. With the aid of the data (e.g., obtained via simulation), the navigation system 14 can then navigate a trackable instrument such that a predetermined amount of bone and/or cartilage material is removed from the pelvis and/or from the femur.

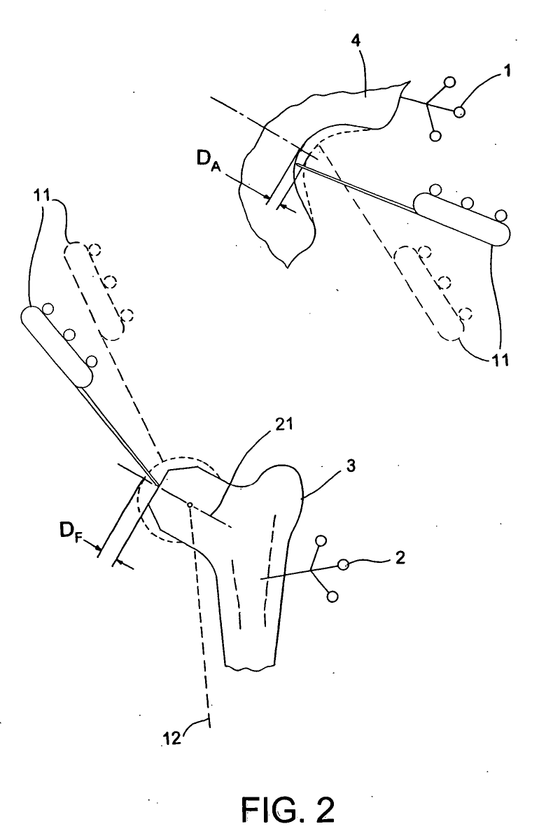

FIG. 2 shows a hip 4 and a femur 3 (in particular a hip joint cavity of the hip 4 and the head of the femur 3) on which markers 1, 2 are arranged and whose contour or surface is registered using a pointer 11. The pointer 11, as it registers the original shape of the hip joint cavity and the head of the femur 3, can be seen by the broken line, as well as the original contour of the hip 4 and/or the hip joint cavity and the femur 3 and/or the head of the femur 3. Furthermore, FIG. 2 shows the head of the femur 3 after the bone and/or cartilage material has been removed, as well as the difference D_{F} between the original contour of the femoral head and its contour after the bone and/or cartilage material has been removed, and the pointer 11 as it registers the new contour. FIG. 2 likewise shows the new contour of the hip joint cavity of the hip 4 after the bone and/or cartilage material has been removed, as well as the difference D_{A} between the original and new contour of the hip joint cavity, and the pointer 11 as it registers the new contour or new shape of the hip joint cavity after the bone and/or cartilage material has been removed.

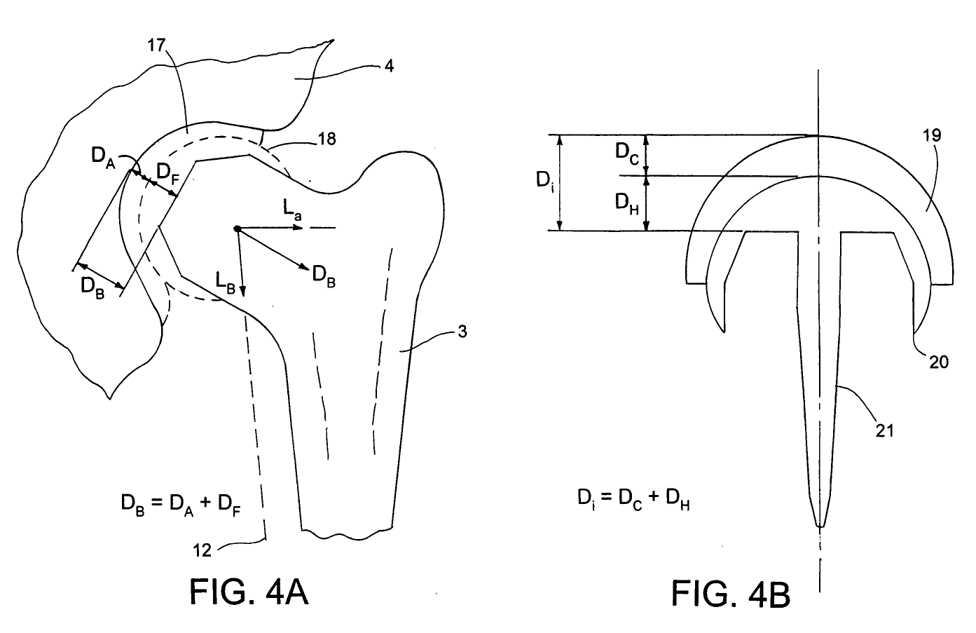

The differences D_{F} and D_{A}, for example, can be transmitted via a wired or wireless connection from the data detection system (e.g., the cameras 16 and pointer 11) to the computational unit and/or to the navigation system 14. The data can be further processed and compared in the computational unit and/or navigation system 14 with known bone and/or cartilage contour data, such as known differences D_{F} and D_{A}, which can be stored in the database 15, for example, to enable selection of at least one suitable hip joint prosthetic element from the database 15. In said comparison, for example, hip joint prosthetic elements can be selected that replace, approximate or imitate the removed bone and/or cartilage material. In particular, a prosthetic cavity 19 as shown in FIG. 4B can be selected, wherein a thickness D_{C} best approximates the thickness D_{A} of the removed bone and/or cartilage material of the hip 4 or the thickness D_{C} at least approximately corresponds to the thickness D_{A}. Preferably, the prosthetic head 20 is selected together with the corresponding prosthetic shaft 21, wherein the thickness D_{H} of the prosthetic head 20 at least approximately corresponds to or at least approximately matches or approximates the thickness D_{F} of the bone and/or cartilage material removed from the femur 3.

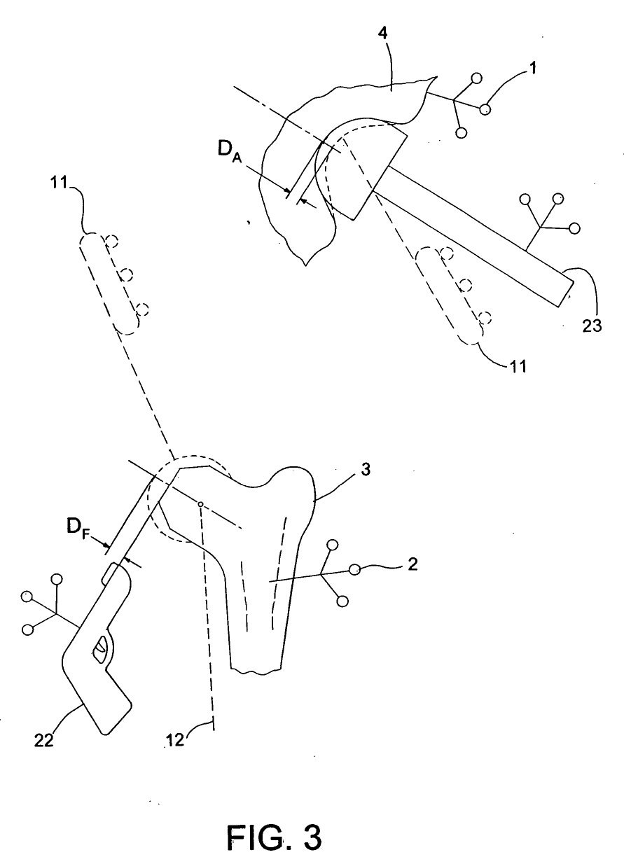

FIG. 3 shows instruments 22, 23 for removing bone and/or cartilage material, each of which can be provided with markers, such as a reference star or the like, and thus can be navigated and/or positioned by the navigation system 14. For example, data or information on a particular amount of bone and/or cartilage material can be transferred to the navigation system 14 or can be known to the navigation system 14, such that with the aid of the navigation system 14, the instruments 22, 23 can be navigated or moved. The navigation or movement is performed such that the desired amount of bone and/or cartilage material can be removed to achieve a desired or input differences D_{F} and D_{A} between the old and the new contour of the hip joint cavity and/or the old and the new contour of the femoral head. The instruments 22, 23 also can be moved such that differences D_{F} and D_{A} are achieved that are necessary due to individual anatomical circumstances (e.g., a particular build of the patient) or medical circumstances (e.g., to enable the patient's health to improve) for example. In particular, the instruments 22, 23 can be moved by a surgeon or automatically moved with the aid of the data from the navigation system 14.

The computational unit and/or the navigation system 14 can select at least one hip joint prosthetic element, such as a prosthetic head 20, a prosthetic shaft 21 and/or a prosthetic cavity 19, from a number of hip joint prosthetic elements stored in the database 15, for example. The selection can be performed by comparing the detected bone and/or cartilage contour data and ascertaining the most suitable prosthetic element, such that the thickness D_{C} of the prosthetic cavity 19 approximately corresponds to the difference D_{A} between the old and the new contour of the hip joint cavity and/or such that the thickness D_{H} of the prosthetic head 20 approximately corresponds to the difference D_{F} between the old and the new contour of the femoral head. The selected hip joint prosthetic element or hip joint prosthetic elements then can be output on the display 14b, for example. Exemplary hip joint prosthetic elements are shown in FIG. 4B, wherein the hip joint prosthetic formed from the hip joint prosthetic elements include a prosthetic cavity 19 and an anchoring element comprising a prosthetic head 20. The prosthetic as a whole, as shown in FIG. 4B, can include two parts, for example, such as the prosthetic cavity 19 and the anchoring element including the prosthetic head 20.

The prosthetic also can include three or more parts, such as a prosthetic cavity 19, which can be mounted in the hip joint cavity and connected to the prosthetic head 20. The prosthetic head 20 in turn can be connected to an anchoring element, such as a prosthetic shaft 21, which, for example, can be inserted and cemented into the femur 3.

FIG. 4A shows the femur 3 as well as the prosthetic and the hip 4 after an operation, wherein the femur 3 and the hip 4 are connected such that the difference D_{F} in the contour of the femur 3 before and after removing bone and/or cartilage material is at least approximately completely compensated for by the prosthetic head 20. The difference in the hip 4 or hip joint cavity before and after removing bone and/or cartilage material is at least approximately completely compensated for by the prosthetic cavity 19.

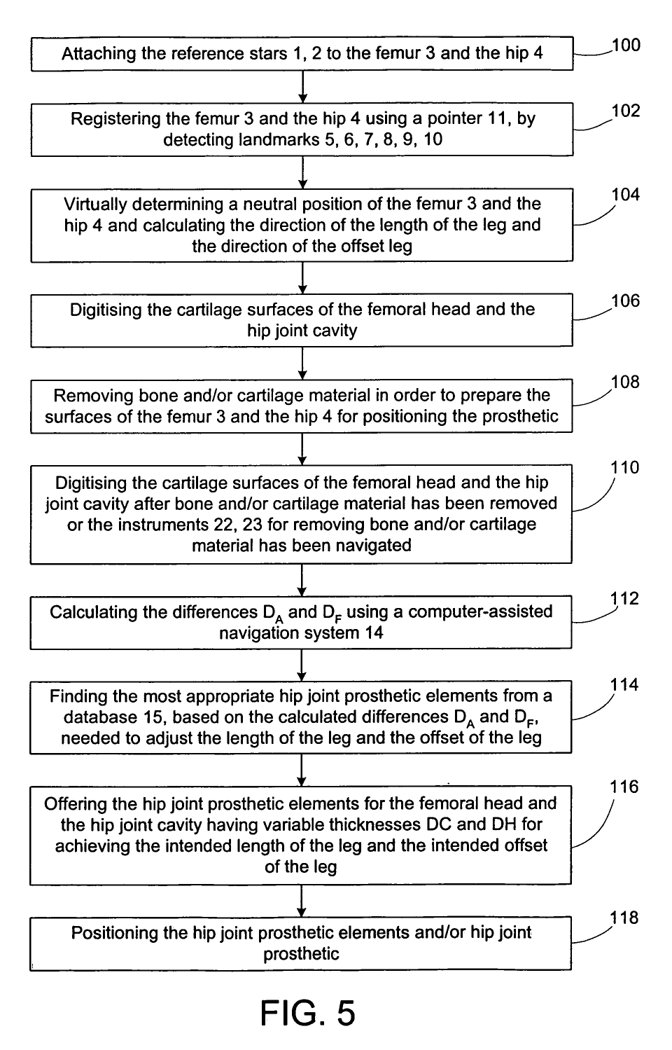

FIG. 5 shows a flow diagram of an embodiment of the method. In a first step 100, reference stars 1, 2 are arranged on or attached to the femur 3 and the hip 4, wherein at step 102 landmark points 5, 6, 7, 8, 9, 10 are then detected using a pointer 11, and the femur 3 and the hip 4 are registered. At step 104, a neutral position of the femur 3 and the hip 4 then is virtually defined or ascertained, for example, in the computational unit and/or the navigation system 14, and the direction of the leg length and the direction of the leg offset are calculated. In step 106, the cartilage contours or cartilage surfaces of the femoral head and the hip joint cavity are digitized, and at step 108 bone and/or cartilage material of the femur 3 and the hip 4 is removed to ensure that the hip joint prosthetic is properly positioned. The bone and/or cartilage material also can be removed using instruments 22, 23, which are navigated by the navigation system 14. After the bone and/or cartilage material has been removed, the cartilage contours or cartilage surfaces of the femoral head and the hip joint cavity is again digitized at step 110, and at step 112 the differences D_{A} and D_{F} between the contours of the hip joint cavity and the femoral head are calculated before and after removing the bone and/or cartilage material. Then, at step 114, by taking into account the calculated differences D_{A} and D_{F}, the most appropriate hip joint prosthetic elements can be sought in a database 15, wherein the navigation system 14 and/or the computational unit finds and outputs the hip joint prosthetic elements whose thicknesses D_{C} and D_{H} best approximate the calculated differences D_{A} and D_{F}, as indicated at step 116. Thus, the previous leg length and/or the previous leg offset can be obtained or restored. Further, the calculated or desired leg length and/or the calculated or desired leg offset can be established. The output or selected hip joint prosthetic elements or the hip joint prosthetic including the selected hip joint prosthetic elements are then positioned or implanted at step 118, e.g., manually by a surgeon or automatically by a robot, wherein the selected prosthetic cavity is positioned in the hip joint cavity and the selected prosthetic shaft and/or the selected prosthetic head is positioned in the femur.

Moving to FIG. 6, an exemplary computational unit 14a for executing a computer program in accordance with the present invention is illustrated. The computational unit 14a includes a computer 20 for processing data. A keyboard 24 and pointing device 26 may be used for data entry, data display, screen navigation, etc. The keyboard 24 and pointing device 26 may be separate from the computer 20 or they may be integral to it. A computer mouse or other device that points to or otherwise identifies a location, action, etc., e.g., by a point and click method or some other method, are examples of a pointing device. Included in the computer 20 is a storage medium 28 for storing information, such as application data, screen information, programs, etc. The storage medium 28 may be a hard drive, for example. A processor 30, such as an AMD Athlon 64® processor or an Intel Pentium IV® processor, combined with a memory 32 and the storage medium 28 execute programs to perform various functions, such as data entry, numerical calculations, screen display, system setup, etc. A network interface card (NIC) 34 allows the computer 20 to communicate with external devices.

The actual code for performing the functions described herein can be easily programmed by a person having ordinary skill in the art of computer programming in any of a number of conventional programming languages based on the disclosure herein. Consequently, further detail as to the particular code itself has been omitted for sake of brevity.

Although the invention has been shown and described with respect to a certain preferred embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. A method for selecting a hip joint prosthetic element from a number of hip joint prosthetic elements, comprising:
detecting bone and/or cartilage contour data of a pelvis and/or a femur; and
selecting at least one hip joint prosthetic element based on the detected bone and/or cartilage contour data.

2. The method as set forth in claim 1, wherein the hip joint prosthetic element is at least one of a prosthetic head or a prosthetic cavity.

3. The method as set forth in claim 1, further comprising attaching trackable markers to the hip and/or femur before the bone and/or cartilage contour data are detected.

4. The method as set forth in claim 1, wherein the bone and/or cartilage contour data are detected using at least one of a pointer, a computer tomography method, a nuclear spin tomography method, an ultrasound method, a positron emission tomography method (PET) or a single photon emission computed tomography method (SPECT).

5. The method as set forth in claim 1, further comprising detecting a first set of bone and/or cartilage contour data before and/or during a hip joint operation.

6. The method as set forth in claim 5, further comprising detecting a second set of bone and/or cartilage contour data while bone and/or cartilage material of the hip and/or the femur is being removed and/or after it has been removed.

7. The method as set forth in claim 6, wherein the step of selecting at least one hip joint prosthetic element includes selecting at least one hip joint prosthetic element based on the first set of bone and/or cartilage contour data and on the second set of bone and/or cartilage contour data.

8. The method as set forth in claim 7, wherein the step of selecting includes selecting the hip joint prosthetic element such that a predetermined leg length and/or a predetermined leg offset is achieved.

9. The method as set forth in claim 1, further comprising providing the detected bone and/or cartilage contour data to a navigation system, wherein said navigation system calculates a neutral standing position of a person from the detected and/or registered bone and/or cartilage contour data.

10. The method as set forth in claim 1, further comprising using the detected bone and/or cartilage data to navigate at least one instrument for removing bone and/or cartilage material.

11. A computer program which, when it is running on a computer or is loaded onto a computer, performs the method as set forth in claim 1.

12. A program storage medium or a computer program product comprising the computer program as set forth in claim 11.

13. A device for selecting at least one hip joint prosthetic element from a number of hip joint prosthetic elements, comprising:
a data detection system for detecting bone and/or cartilage contour data;
a computational unit for processing the detected bone and/or cartilage contour data, wherein the computational unit is communicatively coupled to the data detection system; and
a database in which characteristic data of a number of hip joint prosthetic elements are stored, wherein the computational unit selects at least one hip joint prosthetic element from the database based on the detected bone and/or cartilage contour data.

14. The device as set forth in claim 13, wherein the data detection system comprises:
at least one pointer that includes trackable markers;
at least one camera;
an instrument for removing bone and/or cartilage material, said instrument including trackable markers; and
at least one of a computer tomograph, a nuclear spin tomograph, an ultrasound tomograph, a positron emission tomograph, or a SPECT tomograph.

15. The data detection system of claim 14, wherein the camera is an infrared camera and/or infrared lamps.

16. The device as set forth in claims 13, further comprising
a data output device for outputting the bone and/or cartilage contour data and/or the selected hip joint prosthetic element; and
a data input device for inputting data to be stored in the database and/or data to be processed in the computational unit.

17. The device as set forth in claim 16, wherein the data output device is a screen, and the data input device is a keyboard and/or a scanner.

18. The device as set forth in claim 16, wherein the data input via the data input device includes hip joint prosthetic element data and/or bone and/or cartilage contour data.

19. The device as set forth in claim 13, further comprising a navigation system, wherein the navigation system can navigate at least one trackable instrument for removing bone and/or cartilage material via the detected bone and/or cartilage contour data.

20. The device as set forth in claim 13, further comprising a positioning device for positioning the selected hip joint prosthetic element.

21. A hip joint prosthetic element comprising a partially spherical surface element having an outer contour and an inner contour, wherein a diameter of the outer contour and/or inner contour can be varied by removing or adding an additional element.

22. The hip joint prosthetic element of claim 21, wherein the diameter can be varied between a range of about plus or minus 10 mm.

23. The hip joint prosthetic element as set forth in claim 21, wherein the additional element can be inserted into or removed from the inner contour of the hip joint prosthetic element.

24. The hip joint prosthetic element of claim 23, wherein the additional element is a prosthetic head that includes a convex surface element, or a prosthetic cavity that includes a concave surface element,

25. The hip joint prosthetic element as set forth in claim 23, wherein the additional element is placed on the outer contour of the hip joint prosthetic element and/or can be removed from the outer contour of the hip joint prosthetic element.

26. A hip joint prosthetic element comprising a prosthetic shaft that can be inserted into a bone, wherein a length of the prosthetic shaft can be varied by removing an additional element from the prosthetic shaft of the hip joint prosthetic element or by inserting an additional element into the prosthetic shaft of the hip joint prosthetic element.

27. The hip joint prosthetic element of claim 26, wherein the length of the prosthetic shaft can be varied between about plus or minus 10 mm.

28. An additional element for insertion into or onto an outer and/or inner contour of a hip joint prosthetic element or for being removed from the outer and/or inner contour of the hip joint prosthetic element.
